(19) United States
(12) Patent Application Publication
(10) Pub. No.: US 2004/0013713 A1
(43) Pub. Date: Jan. 22, 2004

(54) Skin conditioning article

(76) Inventors: Craig J. Berry, Westport, CT (US); Kim R. Berry, Westport, CT (US)

Correspondence Address: Geza C. Ziegler, Jr., PERMAN & GREEN, LLP, 425 Post Road, Fairfield, CT, 06824, US

(21) Appl. No.: 10/371,358

(22) Filed: Feb. 20, 2003

Related U.S. Application Data
(60) Provisional application No. 60/359,015, filed on Feb. 20, 2002.

(51) Int. Cl. A61K 9/70

(52) U.S. Cl. 424/443

(57) ABSTRACT

A conditioning article that has a first substrate coated on one side with a cosmetic formulation and a second substrate coated on one side with a cosmetic formulation. The first and second substrates are joined together along corresponding regions of each of the first and second substrates with the coated sides of each substrate facing each other. A cavity is formed between the first and second substrates into which a hand or other body part can be inserted when used. An exterior portion of the article remains substantially dry when the cosmetic formulation in the interior portion of the article is activated by a user for use.

### CROSS-REFERENCE TO RELATED APPLICATIONS

This application claims priority from U.S. Provisional Application No. 60/359,015, filed Feb. 20, 2002.

### BACKGROUND OF THE INVENTION

1. Field of the Invention

The present invention relates to products for treatment of dry skin, particularly dry skin on the hands and face. and, more particularly, to a skin conditioning article.

2. Brief Description of Related Developments

The problem of dry skin has today, become almost universal. There are a multitude of skin care products on the market, including various types of creams, lotions and oils. With most lotion/cream products for conditioning for example the hands or the feet, it is necessary to apply the substance and usually leave it on all day. With greasy formulations, this leads to inadvertent soiling of papers that may be handled, as during office work. Products applied at bed time tend to be rubbed off onto the linens, as the user continuously and unconsciously shifts position during sleep.

Gloves or mitts that can be placed over the hands, such as for example U.S. Pat. No. 5,869,072, the disclosure of which is incorporated herein by reference in its entirety, are single layer and require the application of water for activation on both the interior and exterior surfaces. Generally, the gloves require repeated moisturization in order to facilitate the moisturization of the hand and are only meant to be worn for relatively short periods of time, such as 30-90 minutes.

### SUMMARY OF THE INVENTION

The present invention is directed to a conditioning article. In one embodiment the conditioning article has a first substrate coated on one side with a cosmetic formulation and a second substrate coated on one side with a cosmetic formulation. The first and second substrates are joined together along corresponding regions of each of the first and second substrates with the coated sides of each substrate facing each other. A cavity is formed between the first and second substrates into which a hand or other body part can be inserted when used. An exterior portion of the article remains substantially dry when the cosmetic formulation in the interior portion of the article is activated by a user for use.

In another aspect the present invention is directed to a skin conditioning article. In one embodiment, the skin conditioning article comprises a pair of layers of a non-woven material that are coated on an interior surface with one or more cosmetic formulations. A bond is formed along at least a perimeter of each layer and the pair of layers are joined together along at least three sides of the pair of layers to form an opening along one side. The opening is adapted to allow a user to insert a body extremity into the opening. The pair of layers are substantially dry and an outer surface of each layer remains dry and free from the cosmetic formulation on an interior surface of each of the pair of layers when the skin conditioning article is used.

### BRIEF DESCRIPTION OF THE DRAWINGS

The foregoing aspects and other features of the present invention are explained in the following description, taken in connection with the accompanying drawings, wherein:

FIG. 1 is a perspective view of one embodiment of a skin conditioning article incorporating features of the present invention.

FIG. 2 is an illustration of one embodiment of a skin conditioning mitt incorporating features of the present invention.

FIG. 3 is a block diagram of one embodiment of an apparatus that can be used to make the present invention.

FIG. 4 is a continuation of the apparatus illustrated in FIG. 3.

FIG. 5 is a block diagram of one embodiment of an apparatus used to provide different products and/or coloring in an embodiment of the article illustrated in FIG. 1.

FIG. 6 is a perspective view of another embodiment of a conditioning article incorporating features of the present invention.

### DETAILED DESCRIPTION OF THE PREFERRED EMBODIMENT

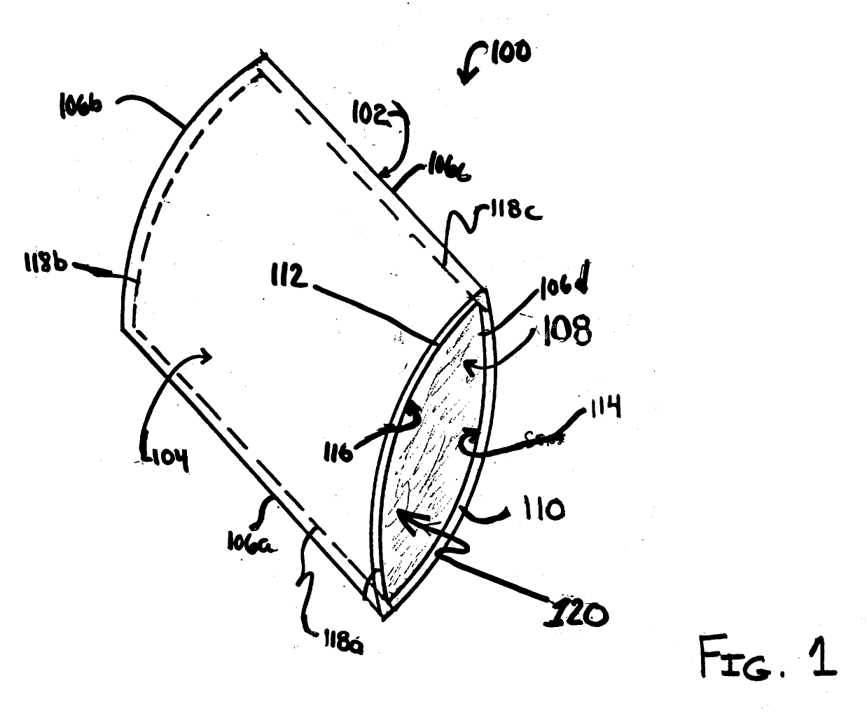

Referring to FIG. 1, a perspective view of an article 100 incorporating features of the present invention is illustrated. Although the present invention will be described with reference to the embodiment shown in the drawings, it should be understood that the present invention can be embodied in many alternate forms of embodiments. In addition, any suitable size, shape or type of elements or materials could be used.

As shown in FIG. 1, the article 100 generally comprises at least two, layers, pieces or sections 110, 112 of a material that are joined or bonded together along, for example, an outer perimeter of each of three sides 106a, 106b and 106c, leaving an opening 108 at one end. The material or materials generally comprise a non-woven cloth, although other materials that are adapted to be coated with a slurry can be used. As shown in FIG. 1, the two layers or materials 110, 112 can be joined together along sides 106a, 106b, 106c, to leave an opening 108 along the unjoined side 106d. Alternatively, any suitable number of sides can be joined. Although the reference is made to two sections or layers of a material, it should be noted that each section 110, 112 could each comprise a different material, resulting in two different materials being bonded together as described. In alternate embodiments, each section 110, 112 could be made up of one or more different materials. Generally, each layer 110, 112 comprises a non-woven material that is adapted to be coated. In alternate embodiments, each layer 110, 112 can comprised any suitable material can be used that can be coated with any one of a number of body cleansing or conditioning agents or materials which can then be applied to parts of the body. It is a feature of the present invention to use the article to apply various skin cleansing or conditioning agents to parts of the body, such as for example the hands and feet. The article remains substantially dry and the formulation or product that is to be applied to the skin remains in an inactive state until the article is placed over the hand or foot.

The opening 108 is generally large enough to allow the insertion of a body part, such as for example a hand or a foot, to be inserted therein. The cavity 120, formed by the two sections 110, 112, has two interior surfaces, 114 and 116. Each interior surface or layer 114, 116 is adapted to hold a conditioning material or materials, while the respective outer surface, 102, 104 of each layer 110, 112, does not. When for example, a hand is inserted into the article 100, the hand comes into contact with the conditioning material or materials. The exterior surfaces 102, 104 of the article 100 remain substantially free from the material(s) on the interior surfaces 114, 116.

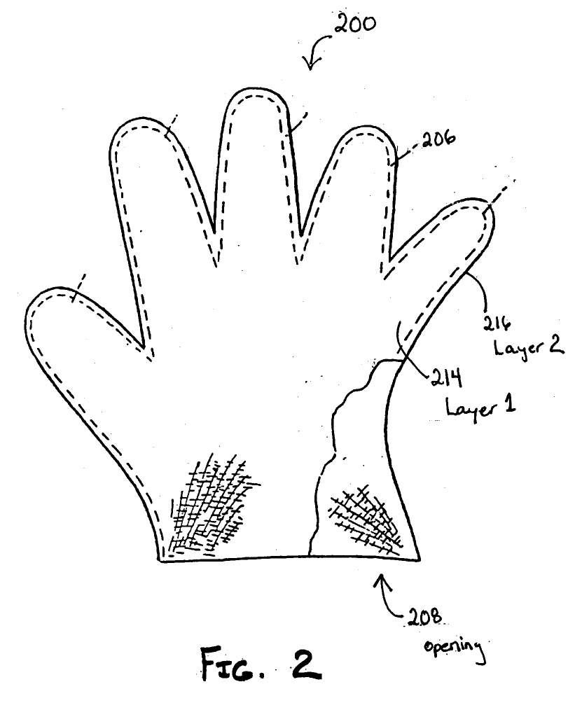

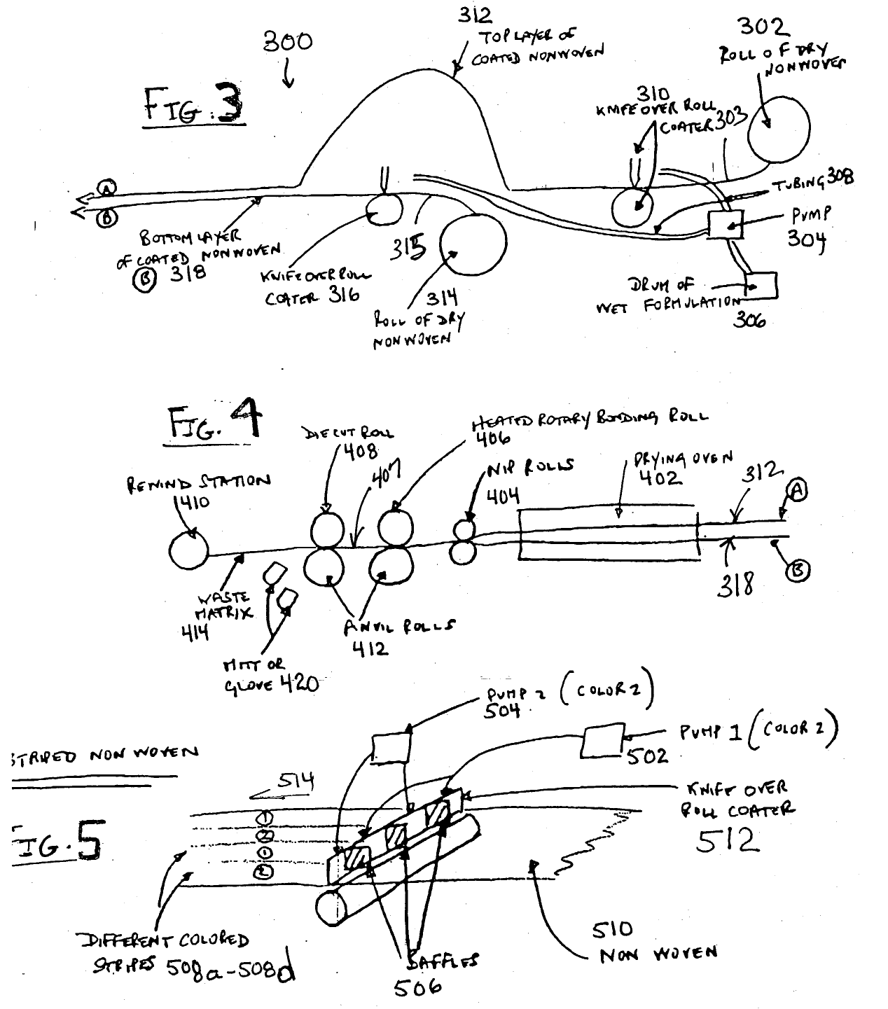

Referring to FIG. 2, in one embodiment, the present invention is generally directed to a non-woven mitt or glove 20 that is coated on the inside for use as a skin conditioning article. The article 200, in the shape of a glove or mitt, is coated on the inside with a skin conditioning product and the outside surfaces, represented by 214, 216, remain essentially dry or free of the product or material that is on the inside. The article 200 of FIG. 2 is adapted to be placed over the hand and left in place for a period of time in order to achieve a desired skin conditioning effect. Although not illustrated, the article 200 could also be formed so that it fits over a foot. The article 200 can also be suitably shaped or sized to accommodate any desired or needed configuration. Although the present invention is described as a mitt or a glove shape, it should be understood that any desired shape could be formed, such as for example, a circle, oval, square, or rectangle. A feature of the present invention is that the article can be placed over the hand and used to condition the hand over a certain period of time. For example, a user may place the article over their hand or hands before bed and allow the article to condition the hand overnight. Since the outside of the article is essentially dry and free of product, the user's bed linens for example, will not be contaminated with product.

Generally, as illustrated in FIG. 1, the article 100 comprises two layers or sections 110, 112 of a non-woven cloth-like material. In one embodiment the layers are heat or sonic bondable, although any suitable method for bonding the two layers can be used. Each layer 110, 112 is adapted to be coated. In alternate embodiments, each layer can comprised any suitable material can be used that can be coated with any one of a number of body cleansing or conditioning agents or materials which can then be applied to parts of the body. It is a feature of the present invention to use the articles to apply various skin cleansing or conditioning agents to the body.

Each of the two layers 110, 112 could also include one or more sub-layers of materials or substrates (not shown) Generally, the materials are water insoluble substrates and can include wet extensible portions. The material or materials for each layer 110, 112 should be able to be adapted to be coated and dried with any one of a number of cosmetic or over-the-counter conditioning ingredients or materials. Although cosmetic or over-the-counter formulations are referred to herein, it should be understood that the present invention is not so limited and any desired formulation, compound or product can be used. In one embodiment, the formulation or product comprises a viscous cream that partially penetrates into the non-woven material. The cream can be coated onto the portion(s) of a substrate, or one or more substrates, that comprise the inside layers 114, 116 of the article 100 to form a distinct layer of product. In this way, the product remains contained to the interior 120 of the article 100, while the exterior surfaces 102, 104 are generally free from the product.

If desired, the product can be adapted to be activated by the user when use is desired. For example, in one embodiment, the product can be heat activated so that when the hand is inserted into the article, the body heat activates the conditioning product. Alternatively, any suitable activation means can be used, such as for example water activation. For example, the present invention can be adapted to incorporate a user's favorite cosmetic or over the counter (“OTC”) formulations in a dry, inactive state, that is activated when a liquid such as water is added to the article 100 of FIG. 1. In this manner, the article 100 can be sized, packaged and adapted for ease of use in a variety of different applications, such as for example while traveling. The article 100 would be generally dry while stored, and when removed for use, wetted to activate the formulations in the materials or layers 110, 112 to achieve the desired result.

Examples of some of these products or materials can include, but are not limited to, body, hand, feet and other skin products, conditioners, creams and lotions. As used herein, the term “cosmetic” generally refers to examples of the foregoing formulations and products.

Although the article 100 is generally described herein as being used to deliver a skin conditioning product to the hand, in alternative embodiments the article can be used to deliver any suitable skin product or skin treatment. Referring to FIG. 1 the article 100 is generally formed by sealing the two layers 110, 112 of material(s) around a perimeter along one or more sides 106a-106c, to form a joined or bonded outline of for example, the mitt or glove shape. For this reason, at least the outer layers of the material it is preferable to have sonic and/or heat bondable properties in order to form a desired bond 118a-118c, around portions of the perimeter of the article 100. Examples of such materials or substrates can include, but are not limited to, Rayon and Polypropylene, and Rayon and Polyester. In alternate embodiments, any suitable means can be used to join or bond the two layers, including for example stitching or gluing. It is a feature of the present invention that the two layers remain bonded together even when the article is wetted or submersed in water. In one embodiment, the layers 110 and 112 comprise a material or materials that when heated and melted bonds under a certain degree of pressure to itself. Examples of such materials can include polypropylene. The actual process of bonding the two layers or materials together is not unique to the present invention and will not be described in detail.

In an embodiment where the layers 110, 112 of FIG. 1 include one or more sub-layers, at least the outermost portions of each layer 110, 112 that make contact must have heat bondable properties in order to form a desired bond 118a-118c around portions of the perimeter of the article. Examples of such heat bondable materials or substrates can include, but are not limited to, Rayon and Polypropylene, and Rayon and Polyester. The material can be of any desired thickness and texture in order to accommodate the cosmetic formulation and still be pleasing to the user. The article 100 could include different textures on each outer side 102, 104 of the article 100, or on the interior sides 114, 116 of the article 100.

It should be noted that although the article is generally described herein as a glove or mitt adapted to fit over a hand, the article can be adapted to be placed over any suitable body part. For example, it may be desired to condition the feet, in which case the article could be shaped as a sock type article. Alternatively, it may be desirable to condition or treat a portion of an arm or leg, in which case the article can be sized and shaped to fit over that body portion. The article is designed to stay in position over the hand, different sizes and shapes can be made to accommodate various users or applications. The shape can also be formed in any suitable manner, such as for example a die cut to form a distinct shape. For example, the article could be configured to be in the shape of a cartoon figure, a sports action figure, a flower, or any other user pleasing shape.

The article can be made of any suitable material or materials that can be used to apply the ingredients to the hands and other body parts in an appropriate manner. In one embodiment, a sonic bonding process can be used to bond the layers together. During a sonic bonding process a horn over roll process is used, where the roll has a pattern with a pin outline. Sound waves are used to bond the material or layers.

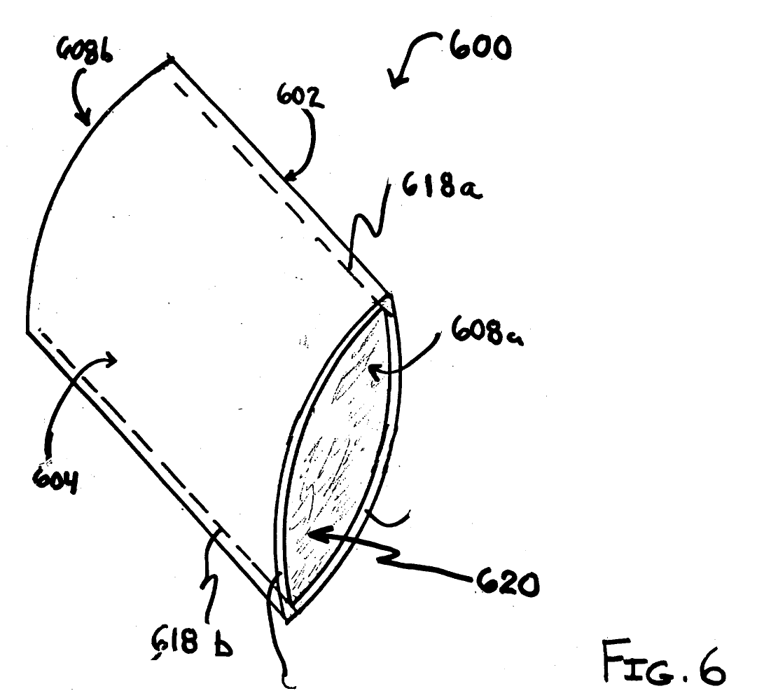

In one embodiment, referring to FIG. 5, different portions or sections 508a-508d, of a layer 510 of non-woven material, can be coated with different formulations. For example, in one embodiment, sections 508a-508d could be coated with a formulation or product in the form of strips along a direction 514 of travel of the layer 510. Although four different sections or “stripes” are shown, it should be understood that any suitable number of different sections can be implemented.

Each section 508a-508d could include a different formulation or product. A different color could be used in each section or “stripe” 508a-508d to distinguish between the different formulations. In alternate embodiments, only selected portions of the article or material 510 might be coated with a cosmetic formulation. Also, different sections of each layer could include different formulations that achieve a different effect on the skin or body.

In one embodiment, the different formulations, or different colors could be divided into any desired number of channels in any suitable fashion prior to passing the material through the knife-over-roll coater. The stripes could be applied to one or both sides of the article. Similarly, in alternatives of this embodiment of the present invention, different formulations or colors could be applied to each of the two layers that form the article.

Each formulation can be pumped into the channels 508a-508d in any suitable fashion prior to passing the material through the knife-over-roll coater 102. For example, pumps 502, 504 could be used to apply the different formulations and/or colors, through baffles 506 to the material 510 as it passes through the knife over roll coater 512. In alternate embodiments, any suitable number of pumps can be used to apply a corresponding number of stripes or sections 508a-508d of formulation. A similar process can be used to apply the mixed coating to the other layer. It should also be understood that in alternative embodiments, different formulations or colors could be applied to each layer of the two layers that form the article 100 of FIG. 1. For example, referring to FIG. 1, one layer 110 could be coated on its interior surface 114 with a first formulation and the interior surface 116 of the second layer 112 can be coated with a second formulation. The first and second formulation could each be colored differently in order for the user to be able to distinguish between the different formulations on each layer. Again, the first layer 110 could include a number of different formulations while the second layer 112 also includes a number of different formulations. The formulations could all be different or some may be the same.

Referring to FIG. 6, in one embodiment the article 600 is only bonded along two-sides, 618a and 618b, in order to create a sleeve-like article. In this embodiment, the opening 608a leads to a sleeve 620 with a similar opening 608b at the opposing end. The article 600 can be placed over an extremity, such as a leg or an arm. The article 600 can be suitably sized in order to accommodate any particular length or diameter. In one embodiment, the article 600 could be a stretchable material that maintains a sufficient tightness around the extremity to keep from moving inadvertently. Also, instead of being bonded on two sides, the article 600 could be bonded along only one edge 618a and include mechanical fasteners, such as for example Velcro or adhesive tape along the other edge 618b to enable securing the article 600 around the extremity. The article 600 could comprise two separate pieces or sections 602, 604, or could comprise a single piece folded over on itself to have two sides or sections 602, 604.

Some features and benefits of the present invention can include the incorporation of the user's favorite cosmetic or over the counter (“OTC”) formulations in a dry, inactive state that is activated when a liquid such as water is added to the article. In this manner, the article can be size, packaged and adapted for ease of use in a variety of different manners, such as for example while traveling. The article would be dry while stored, and when removed for use, wetted to activate the formulations in the materials to achieve the desired result. The article is generally adapted to be a single use, disposable article. The incorporation of the cosmetic or OTC formulation into the non-woven material can generally enhance the performance of the formulation.

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

What is claimed is:

1. A conditioning article comprising:
a first substrate coated on one side with a cosmetic formulation; and
a second substrate coated on one side with a cosmetic formulation, the first and second substrates being adapted to be joined together along corresponding regions of each of the first and second substrates with the coated sides of each substrate facing each other, a cavity being formed between the first and second substrates having an opening into which a hand can be inserted; and
wherein an exterior portion of the article is adapted to remain substantially dry when the cosmetic formulation in an interior portion of the article is activated by a user for use.

2. The conditioning article of claim 1 wherein the first and second substrate each comprise a non-woven cloth-like material.

3. The conditioning article of claim 1 wherein the first and second substrate each include a water insoluble substrate and a wet extensible portion.

4. The conditioning article of claim 1 wherein the article is a single use, disposable article.

5. The conditioning article of claim 1 wherein the article is formed in a shape of a glove which is adapted to fit over a hand.

6. The conditioning article of claim 1 wherein an outer perimeter of each of the first substrate and the second substrate is heat bondable, and wherein when the first substrate and the second substrate are formed and heat is applied, the perimeter of the first substrate and the perimeter of the second substrate are heat bonded.

7. The conditioning article of claim 1 wherein the first substrate is coated with a first cosmetic formulation that is different from a second formulation used to coat the second substrate.

8. The conditioning article of claim 1 wherein the first substrate is divided into sections and a different formulation is coated onto at least one of the sections.

9. The conditioning article of claim 1 wherein the first substrate is coated with at least two cosmetic formulations, each formulation being coated on the substrate adjacent to another formulation.

10. The conditioning article of claim 9, wherein each formulation is coated on the substrate in a form of a stripe, each stripe representing a different formulation.

11. The conditioning article of claim 9, wherein each stripe has a different color.

12. The conditioning article of claim 1 wherein both the first substrate and the second substrate are coated with a respective series of cosmetic formulations, each formulation being coated in a form of a stripe along the first substrate and the second substrate, wherein one formulation is coated adjacent to another substrate.

13. The conditioning article of claim 1 wherein the first substrate and the second substrate comprise a heat-bondable material, and the first substrate and the second substrate are heat-bonded together.

14. The conditioning article of claim 1 wherein the first substrate and the second substrate are adapted to be sonically bondable together.

15. The conditioning article of claim 1 wherein each of the first substrate and the second substrate include a bonding material that is adapted to be heat bondable and sonically bondable on itself.

16. The conditioning article of claim 15 wherein the bondable material is polypropylene.

17. A skin conditioning article comprising:
a pair of layers of a non-woven material that are coated on an interior surface with one or more cosmetic formulations;
a bond along at least a perimeter of each layer, wherein the pair of layers are joined together along at least three sides of the pair of layers to form an opening along one side, the opening adapted to allow a user to insert a body extremity into the opening; and
wherein the pair of layers are substantially dry and an outer surface of each layer remains dry and free from the cosmetic formulation on an interior surface of each of the pair of layers when the skin conditioning article is used.

18. The skin conditioning article of claim 17 wherein the article is in the shape of a mitt that is adapted to fit over a hand.

19. The skin conditioning article of claim 17 wherein the cosmetic formulation on the interior surface of each layer is heat activated, and is adapted to activate when the article is placed over a hand.

20. The skin conditioning article of claim 17 wherein the cosmetic formulation on the interior surface of each layer is water activated, and is adapted to activate when the article is placed over a hand that is moist.
